(19) United States
(12) Patent Application Publication
(10) Pub. No.: US 2008/0095744 A1
(43) Pub. Date: Apr. 24, 2008

(54) Methods and Compositions for Cytokine Expression and Treatment of Tumors

(76) Inventors: Jacqueline N. Parker, Birmingham, AL (US); Richard J. Whitley, Birmingham, AL (US); James M. Markert, Birmingham, AL (US)

Correspondence Address: GIFFORD, KRASS, SPRINKLE, ANDERSON &;CITKOWSKI, P.C., P.O. BOX 7021, TROY, MI, 48007-7021, US

(21) Appl. No.: 11/718,439

(22) PCT Filed: Nov. 1, 2005
(86) PCT No.: PCT/US05/39291

§ 371 Date: May 14, 2007

Related U.S. Application Data
(60) Provisional application No. 60/624,245, filed on Nov. 2, 2004.

(51) Int. Cl. A61K 39/245; C12N 7/01; A61P 35/00

(52) U.S. Cl. 424/93.2; 435/235.1

(57) ABSTRACT

A conditionally replicating, aneurovirulent recombinant herpes simplex virus is provided that includes a nucleic acid encoding an expressible chemokine. An expression control element is operably linked to the nucleic acid. A therapeutic composition includes a first conditionally replicating, aneurovirulent recombinant herpes simplex virus having a first nucleic acid encoding a first expressible cytokine operatively linked to an expression control element. A second such herpes simplex virus encoding a second expressible cytokine is also provided with a pharmaceutically acceptable carrier. Preferably, the second cytokine is a chemokine. In a particular embodiment, the first expressible cytokine increases availability of an immunoresponsive cell for activation. The activatable cell is CD4+, CD8+, NK, a dendritic cell, or a combination thereof. A second conditionally replicating, aneurovirulent, recombinant herpes simplex virus expresses a second cytokine in the same host cell as the first virus or alternatively, upon expression in a second host cell, resulting in the activation of the immunoresponsive cell. A method of tumor cell growth inhibition is provided that includes introduction of a therapeutically effective amount of an aforementioned therapeutic composition into a tumor of an individual such that two different cytokines are produced within the tumor to enhance the immune response in the individual that inhibits tumor cell growth. The first and second recombinant herpes simplex viruses are administered simultaneously, sequentially. Sequential administration is separated by a time period ranging from a few second to several days.

### RELATED APPLICATION

This application claims priority of U.S. Provisional Patent Application Ser. No. 60/624,245 filed Nov. 2, 2004, which is incorporated herein by reference.

### GRANT REFERENCE

The research carried out in connection with this invention was supported in part by National Cancer Institute grant #CA 071933.

### FIELD OF THE INVENTION

The present invention in general relates to an engineered herpes simplex virus and in particular to a herpes simplex viral vector replicating in and lysing in only dividing cells such as the cells found in tumors.

### BACKGROUND OF THE INVENTION

Eradication of malignancies arising in the brain has proven to be a formidable task. As an example, high-grade malignant gliomas, the most common primary brain tumor, are almost always fatal despite aggressive surgical resection, radiotherapy and chemotherapy. Moreover, the overall five year survival rate for glioblastoma (GBM), the most malignant glioma, is less than 5.5% and the median survival is approximately one year (1).

Because of poor survival of patients with primary intracranial malignancies, novel therapeutic approaches, most notably viral and gene therapy, have been investigated; for reviews see references (2-4). The inventors and others have reported on the efficacy of using neuroattenuated replication-competent herpes simplex viruses (HSV) for the treatment of primary brain tumors. These viruses typically contain one or more mutations within the viral genome, including thymidine kinase (tk) (5), ribonucleotide reductase (UL39) (6, 7), UTPase (8) or γ₁34.5 (9, 10). The γ₁34.5 gene of HSV is present in two copies and located within the inverted repeats of the unique long segment. Mutations within this gene have been shown previously to cause reduction in viral replication and associated neurovirulence of HSV (11). Thus, γ₁34.5-deleted HSV are able to selectively replicate and destroy glioma cells in vivo, without damaging surrounding brain tissue. Moderate increases in long-term survival for engineered HSV-treated versus untreated animals have been reported in both syngeneic and xenogeneic murine tumor models of GBM (5, 9, 10, 12-16). In addition, Phase I studies in humans with malignant glioma have demonstrated that two γ₁34.5-deleted HSV, G207 and 1716 are safe for intracranial inoculation (17-19), albeit at different quantities of inoculated virus. G207, which contains mutations within both copies of the γ₁34.5 gene and in the UL39 gene, has been safely administered at doses up to 3×10⁹ plaque forming units (pfu).

Despite these advantages, existing experimental data suggest that multiple modalities of therapy will be necessary to eradicate malignant tumors of the central nervous system (CNS) as well as those originating outside the brain.

### SUMMARY OF THE INVENTION

A conditionally replicating, aneurovirulent recombinant herpes simplex virus is provided that includes a nucleic acid encoding an expressible chemokine. An expression control element is operably linked to the nucleic acid. A therapeutic composition includes a first conditionally replicating, aneurovirulent recombinant herpes simplex virus having a first nucleic acid encoding a first expressible cytokine operatively linked to an expression control element. A second such herpes simplex virus encoding a second expressible cytokine is also provided with a pharmaceutically acceptable carrier. Preferably, the second cytokine is a chemokine. In a particular embodiment, the first expressible cytokine increases availability of an immunoresponsive cell for activation. The activatable cell is CD4+, CD8+, NK, a dendritic cell, or a combination thereof. A second conditionally replicating, aneurovirulent, recombinant herpes simplex virus expresses a second cytokine in the same host cell as the first virus or alternatively, upon expression in a second host cell, resulting in the activation of the immunoresponsive cell.

A method of tumor cell growth inhibition is provided that includes introduction of a therapeutically effective amount of an aforementioned therapeutic composition into a tumor of an individual such that two different cytokines are produced within the tumor to enhance the immune response in the individual that inhibits tumor cell growth. The first and second recombinant herpes simplex viruses are administered simultaneously, sequentially. Sequential administration is separated by a time period ranging from a few second to several days.

### BRIEF DESCRIPTION OF THE DRAWINGS

FIG. 1 is a schematic representation of various cytokine expressing HSV. Line 1 schematically illustrates the prior art HSV-1 (F) Δ305 genome containing a 501 base pair dilution within the thymidine kinase (tk) gene, as indicated by the Δ symbol. U_{L} and U_{S} represent unique long and short sequences, respectively, while the inverted repeat sequences are indicated by a, b and c, with subscript n and subscript m representing variable numbers of a sequences. aₗ and aₛ represent flanking sequences to the U_{L} and U_{S} terminal repeats. Line 2 schematically illustrates the prior art sequence arrangement of the recombinant HSV R3659. In the R3659 construct, the BstEII-StuI fragment within the γ₁34.5 gene is replaced by a chimeric α27-TK gene in the inverted sequences (shown above) and b′a′ (not shown) flanking the U_{L} sequence. Line 3 schematically illustrates the prior art regions in a recombinant mIL-12 expressing HSV M002 (tk+). Line 4 schematically illustrates the sequence arrangement of the relevant regions of a recombinant mCCL2 expressing HSV M010. Egr-1p denotes the murine early-growth response-one promoter having transcriptional control over mIL-12 and mCCL2 expression depicted in lines 3 and 4, respectively.

FIG. 2a depicts a list of five experimental tumor injection dose groups, and 2b is a timeline indicating the total plaque forming units (pfu) administered as a function of time. With regard to Group V including the combination of vector M002 and M010F, a dose of each vector is delivered to cumulatively achieve dosing denoted in FIG. 2b.

FIG. 3 is a graph noting average tumor volume in cubic millimeters as a function of time for each of the injection volume vectors detailed in FIG. 2. The arrows along the time scale correspond to the pfu doses denoted in FIG. 2b.

FIG. 4a is a bar graph denoting the relative amount of interferon gamma produced within tumors treated according to the dosing regime shown in FIGS. 2a and 2b. Control (Group I synonymously noted as DMEM) showed no production and is excluded from the graph.

FIG. 4b is a bar graph denoting the relative amount of interleukin-12 produced within tumors treated according to the dosing regime shown in FIGS. 2a and 2b. Control (Group I synonymously noted as DMEM) showed no production and is excluded from the graph.

FIG. 4c is a bar graph denoting the relative amount of chemokine CCL2 produced within tumors treated according to the dosing regime shown in FIGS. 2a and 2b. Control (Group I synonymously noted as DMEM) showed no production and is excluded from the graph.

FIG. 5 is a series of immunohistological micrographs identifying inflammatory cell infiltrates. Serial microtomed sections were reacted with rat monoclonal antibodies to CD4+ in the left two columns or CD8+ in the right two columns as detected with horseradish peroxidase labeled anti-rat antibody for tumors excised at Days 6 or 13 for each of the non-control groups denoted in FIG. 2a.

### DETAILED DESCRIPTION OF THE INVENTION

Oncolytic HSV are described herein as vectors for medical therapy and expression of therapeutic compounds. These HSV, which contain identical deletions within both copies of the γ₁34.5 gene, retain the ability to replicate in, and lyse rapidly dividing cells, such as found in tumors, but are unable to replicate in post-mitotic cells, such as those found in normal adult CNS. These conditionally replication competent HSV have been engineered to express foreign genes designed to augment their antitumor effects. Initially, HSV mutants that express interleukin-4 (IL-4) or IL-10 are evaluated in an orthotopic model of murine glioblastoma utilizing syngeneic GL-261 tumors implanted into immunocompetent C57BL/6 mice (14). In this model, treatment with IL-4-expressing HSV increased survival over treatment with HSV alone, suggesting that cytokine gene therapy may mediate enhanced tumor-specific killing. IL-4 gene therapy has been shown to enhance anti-glioma effects in several gene therapy models (20-22). Such effects are TH2-mediated and have been attributed to CD4+ lymphocytes and other effector cells such as eosinophils (23). While IL-4 is effective in these animal models, it did not result in a more durable antitumor effect that would be achieved through generation of a TH-1 response, including the induction of a memory response against tumor cells. Towards this end, a conditionally replication competent HSV-1 expressing IL-12 (M002) has been constructed and characterized. IL-12 is a cytokine that mediates the TH1-type immune response, stimulates NK cell activity, and has antiangiogenic (24-26) and antitumor properties in a variety of models (25, 27). Further, a γ₁34.5-deleted HSV-1 expressing murine interleukin 12 (M002) prolongs survival of immunocompetent mice in an experimental intracranial murine model of neuroblastoma (28). The inventors have previously demonstrated that recombinant HSV that express murine IL-4 (R8306) or murine IL-12 (M002) could significantly improve survival when injected into tumors implanted in brains of immunocompetent mice in a syngeneic murine model (14, 28).

Improved anti-tumor compositions and methods are provided by the present invention. In particular, compositions and methods are provided which combine attraction of immune cells to a desired site and their activation with anti-tumor properties of HSV infection.

In one embodiment of an inventive composition, a recombinant herpes simplex virus also is provided which includes a nucleic acid encoding a cell attractant which is a chemokine.

Chemokines are attractants for particular cell types including neutrophils, monocytes, dendritic cells, T-lymphocytes, natural killer cells, B-lymphocytes, basophils, and eosinophils. Chemokines are characterized by conserved cysteine residues and are generally classified as one of four chemokine types: C-C chemokines; C-X-C chemokines, C chemokines; and C-X-X-X-C chemokines. C chemokines are exemplified by lymphotactin, and fractalkine is an example of a C-X-X-X-C (or CX3C) chemokine. C-C chemokines include RANTES, MIP-1α, MIP-1β, MCP-1, MCP-2, MCP-3, MCP-4, eotaxin, I-309, HCC-1, HCC-2, and HCC-4.

Chemokines of the C-X-C type can be further divided into two groups: those including the three amino acid sequence ELR (glutamic acid-leucine-arginine) preceding the first cysteine residue near the amino terminus and those lacking this ELR motif. CXC chemokines having the ELR motif include IL-8, GROα, GROβ, GROγ, PF4, ENA-78, GCP-2, and NAP-2. CXC chemokines having the ELR domain primarily act on neutrophils. CXC chemokines lacking the ELR motif include IP-10, Mig, I-TAC, SDF-1, and BCA-1.

C-X-C chemokines lacking an ELR motif, CC, and C chemokines chemoattract and activate a variety of cells including monocytes, dendritic cells, T lymphocytes, natural killer cells, B lymphocytes, basophils, and eosinophils.

Detailed descriptions of chemokines and their nomenclature are found in standard texts and reviews, including, for instance: Olson, T. S. and Ley, K., (2002) Am. J. Physiol. Regulatory Integrative Comp. Physiol., 283:R7-R28; Murdoch, C. & A. Finn (2000) Blood 95:3032; Rossi, D. & A. Zlotnik (2000) Annu. Rev. Immunol. 18:217; Sallusto, F. et al. (2000) Annu. Rev. Immunol. 18:593; and Zlotnik, A. & O. Yoshie (2000) Immunity 12:121.

In a preferred embodiment, the chemokine encoded by an inventive HSV is CCL2.

CCL2, formerly called monocyte chemoattractant protein-1 or MCP-1, is an example of a compound that increases the antitumor effects of IL-12 and which is particularly effective, a fact which may be due to its ability to stimulate chemotaxis of monocytes and numerous T lymphocyte subsets. CCL2 is a member of the C-C chemokine family and stimulates chemotaxis of monocytes, CD4+ and CD8+, memory T lymphocytes, and dendritic cells in vitro (29, 30). A differential effect on macrophages of CCL2 stimulation in the presence of IL-12 has been shown (31, 32).

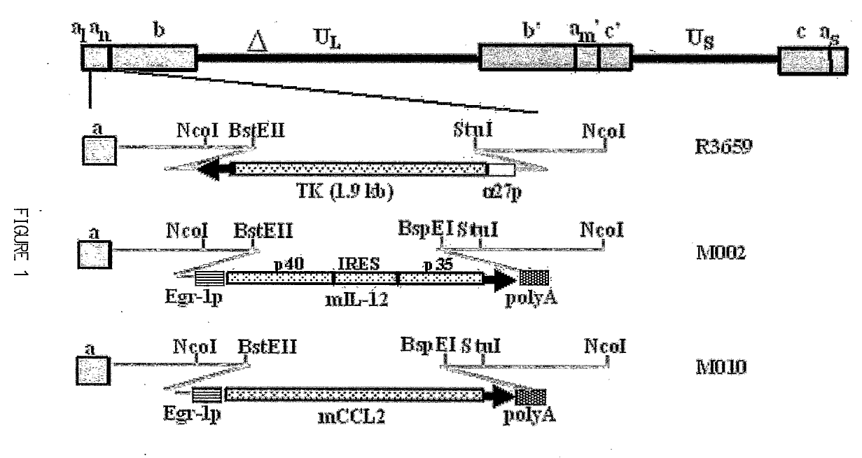

An exemplary virus encoding CCL2, M010, is constructed as described in Examples herein and the genomic organization of M010 is illustrated in FIG. 1 at line 4 as are comparative prior art viruses R3659 (line 2) and M002 (line 3). The base common prior art virus construct is also shown in FIG. 1, line 1.

The inventors find that treatment with a herpes simplex virus expressing a chemokine in combination with a herpes simplex virus expressing a non-chemokine cytokine enhances tumor killing when compared with tumors treated with either virus alone. Thus, in one embodiment, an inventive composition for inhibiting growth of a tumor includes a first HSV expressing a chemokine and a second HSV expressing a cytokine.

In a particular embodiment, an inventive composition includes a first recombinant herpes simplex virus including a nucleic acid encoding a chemokine that upon expression under the control of an expression control element, the chemokine is active in increasing the availability of a cell for activation by a cytokine. In particular, a preferred chemokine attracts a CD4+, CD8+, NK or a dendritic cell for activation by a cytokine. Various chemokines are known to be capable of attracting such cells, including a C-X-C chemokine lacking an ELR motif, a CC chemokine, or a C chemokine. Exemplary chemokines of this type include CCL2, IP-10, Mig, I-TAC, SDF-1, BCA-1, RANTES, MIP-1α, MIP-1β, MCP-2, MCP-3, MCP-4, eotaxin, I-309, HCC-1, HCC-2, HCC-4 and lymphotactin.

Further, an inventive composition contains a second recombinant herpes simplex virus which includes a second nucleic acid encoding a cytokine capable of activating immune-responsive CD4+, CD8+, NK, or dendritic cells. Various cytokines are known to be capable of activating a CD4+, CD8+, NK, or dendritic cell, including for instance IL-12, GM-CSF, cytosine deaminase, IL-1α, IL-1β IL-2, IL-3, IL-4, IL-5, IL-6, IL-7, IL-10, IL-15, IL-23, IL-24, IFN-α, IFN-β, IFN-γ, TNFα and TNFβ.

In a preferred embodiment, an inventive composition for inhibiting growth of a tumor includes a first HSV expressing CCL2 and a second HSV expressing IL-12. Exemplary conditionally replication competent, γ₁34.5-deleted virus constructs include M010 as described above, which encodes CCL2 and M002 which encodes IL-12.

In a further embodiment, a chemokine capable of attracting a cell and a cytokine capable of activating the attracted cell are encoded by nucleic acid included in the same virus.

A nucleic acid encoding a cytokine in an inventive virus and composition are operably linked to an expression control element. Preferably a promoter is chosen to be driven by cellular events relevant to the therapy, such as cell division or exposure of the cell to an exogenous induction factor such as tetracycline. For example, a preferred expression control element is a eukaryotic promoter such as egr-1, EF-2, and B-myb, a viral promoter such as CMV, c-myc and intronless c-myc, and a doxycycline-inducible promoter such as tet-on.

In a preferred embodiment, an inventive virus as well as viruses included in an inventive composition is a conditionally replication competent, aneurovirulent HSV. Thus, preferably, an inventive virus is γ₁34.5 deleted. To this end, a nucleic acid encoding a cytokine is preferably inserted in a γ₁34.5 locus of a herpes simplex virus genome as described herein.

An inventive method is provided for inhibiting tumor cell growth which includes the step of introducing a composition as described herein into an individual for treatment of a tumor. In a preferred embodiment, the tumor is a primary brain tumor. Further preferably, the tumor is a glial cell tumor, such as a glioma.

Administration ratios operative herein include intratumoral, intrathecal, and intraventricular. A preferred route of administration is direct, intratumoral injection. Compositions suitable for injection may comprise physiological acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be controlled by addition of any of various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

The terms “individual” and/or “subject” as used herein means any animal including humans. Examples of individuals and/or subjects include humans, rodents, and monkeys.

A “therapeutically effective amount” is an amount of one or more HSV vectors expressing a cytokine, particularly IL-12 and/or CCL2, that when administered to a patient or subject, inhibits tumor growth, causes tumor regression, prevents metastasis or spread of the tumor, prolongs the survival of the subject or patient, and combinations thereof.

The anti-tumor agents of the present invention can be administered to an individual or subject either alone or as part of a pharmaceutical composition of the agents admixed with a pharmaceutically acceptable carrier, diluent, or excipient.

The vector M010 expresses physiologically relevant levels of CCL2, is not neurotoxic, and can be safely inoculated into murine brain at doses of at least up to 5×10⁷ pfu. Co-administration of M010 with M002 in a syngeneic flank tumor model of a nervous system tumor produces statistically significant increased antitumor effects over either virus administered alone. Further, immunohistochemical analysis of HSV-treated flank tumor sections demonstrate a greater influx of CD4+ and CD8+T cells into tumors treated with a combination of M002 and M010 when compared with tumors treated with either virus alone. Thus, combination immunotherapy for treatment of nervous system malignancies enhances the antitumor effects of HSV-derived vectors.

Conditionally replicating, engineered HSV have been shown to be effective anti-neoplastic agents in glioma and other animal tumor models. Both growth inhibition of subcutaneous tumors and improvement in survival of mice implanted with intracranial tumors has been repeatedly demonstrated. However, only a fraction of animals appear to be cured by viral therapy utilizing current γ₁34.5-deleted vectors. To improve the efficacy of this strategy, the inventors evaluate oncolytic γ_{134.5}-deleted HSV expressing a panel of cytokines and chemokines for anti-tumor effects. Replication of HSV-1 in tumor cells is not affected in vitro by foreign gene expression, and tumoricidal effects in vitro are not diminished and may actually be increased.

IL-12 is a heterodimeric cytokine with potent antitumor properties. It is produced by macrophages, monocytes, B cells, dendritic cells, and other antigen presenting cells (27). It acts to enhance the cytolytic activity of natural killer cells and cytotoxic T lymphocytes as well as the development of a T_{H}1-type immune response via the induction of interferon gamma (IFN-γ) secretion. Indeed in flank tumors treated with M002, IFN-γ levels are significantly greater, as compared to levels detected from tumor homogenates from the other treatment groups, as determined by ELISA.

IL-12 also possesses antiangiogenic properties, which likely represents an additional mechanism for its antitumor activity (26, 27). IL-12 has been shown to produce antiglioma immune activity in a murine model (40). Its pro-inflammatory effects that do not rely on an intact IL-2 receptor pathway have been suggested as a potentially important approach for immunotherapy (41).

CCL2 is a C-C chemokine that directs the leukocytes to infiltrate tissues in order to produce an inflammatory response. CCL2 stimulates the influx of CD4+, CD8+, NK and dendritic cells, as well as macrophages. CCL2 has also been shown to bind to the abluminal side of brain microvessels and stimulate trafficking of mononuclear cells into the CNS parenchyma to await further activation (42). While CCL2 levels are often elevated within gliomas, the absolute level of cytokine and chemokine expression can produce differing effects on target cells. Additionally, the production of a T_{H}1 versus T_{H}2 response by CCL2 and its receptor CCR2 are known to depend on the local tissue environment, the timing of CCL2 induction, the type of antigen, route of inoculation/immunization, and the tissue site (31). It has been reported previously that co-delivery of a recombinant adenovirus (rAd) expressing CCL2 into tumor cells with a rAd that expressed HSV-tk demonstrated enhanced antitumor effects in hepatocellular carcinoma (43), as did a more recently described bicistronic rAd that expressed both CCL2 and HSV tk (44).

In one embodiment of an inventive method at least two viruses are administered in a single composition dose in an equivalent ratio. In another embodiment, at least two viruses are administered in a single composition dose in a ratio ranging from 1000:1-1:1000, inclusive, and preferably in a ratio from 50:1-1:50. In a further embodiment, at least two viruses are administered as a sequential dose, that is, sequentially in a ratio ranging from 1000:1-1:1000, inclusive, and preferably in a ratio from 50:1-1:50. In addition, multiple single doses or sequential doses may be administered over the course of a treatment. In general, a total number of plaque forming units administered per single or sequential dose ranges between 1×10²-1×10⁵⁰, inclusive, and preferably 1×10⁴ and 1×10¹⁰.

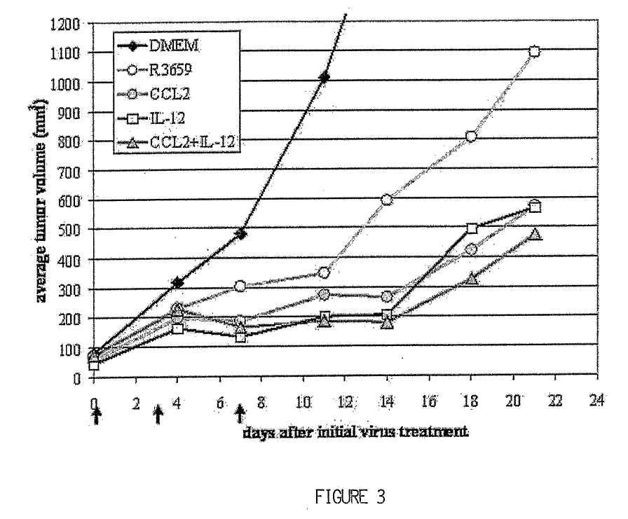

Tumor growth tends to become static or even decrease slightly after each virus dose (see FIG. 3), although the lag period is slightly longer for the Day 7 dose than for the Day 3 dose. This effect is only observed in the groups treated with cytokine-expressing HSV.

Immunohistochemical analysis of Neuro-2a flank tumors treated with a single dose of M002 in combination with M010 reveal a rapid influx of both CD4+ and CD8+ T cells into the tumors by Day 3, which is not observed in any of the other treatment groups. Both cell populations remain consistently elevated, with CD8+ levels higher at the earlier time points (Days 3 and 6) and CD4+ T cell levels higher at the latter time points (Days 10 and 13). Of the single HSV treatment groups, only M002 (IL-12 expressing HSV) treated tumors exhibit similar influx of CD4+ and CD8+ T cell subsets, and only at the last time point assessed (Day 13). In addition, only the M002 in combination with M010 treatment group had significant CD4+ T cell responses still detected at Day 13 after treatment.

The differences between the effector cell populations elicited over time by the different treatment groups may be explained on the basis of differential activation of effector cells infiltrating experimental tumors as has been observed by the inventors (45-48) and others. In one embodiment of an inventive method, specific effector cells, such as CD8+ T cells, macrophages and/or NK cells are targeted to increase anti-tumor activation.

### EXAMPLES

### Example 1

### Cells

Vero cells (American Type Culture Collection [ATCC], Rockville, Md.) are grown and maintained in Minimal Essential Medium (Cellgro, Mediatech Inc., Herndon, Va.) containing 7% fetal bovine serum. The human 143 thymidine kinase minus cells (143tk−, ATCC) are grown in Dulbecco's modified Eagle's medium (DMEM) (Cellgro) supplemented with 10% fetal bovine serum. Rabbit skin cells (originally acquired from Dr. J. McClaren, University of New Mexico, Albuquerque, N. Mex., USA) are maintained in DMEM supplemented with 5% fetal bovine serum. The murine neuroblastoma cell line Neuro-2a (derived from strain A/J mice) is purchased from the ATCC (CCL 131, passage 171) and is maintained in a 50:50 mixture of DMEM and Ham's Nutrient Mixture F-12 (DMEM/F12) supplemented to 2.6 mM L-glutamine and 7% FBS.

### Example 2

### Plasmids and Viruses

HSV-1 (F) strain is a low passage clinical isolate used as the prototype HSV-1 strain in our series (33, 34). Virus R3659 has been described previously (35). Construction of M002, which expresses murine IL-12 (mIL-12) under the transcriptional control of the murine early-growth response-1 promoter (Egr-1), has been described previously (28). The murine clone for CCL2 (monocyte chemotactic protein or MCP-1, homologous to mouse Sig-je) is obtained from ATCC (Rockville, Md.). The HSV shuttle plasmid pRB4878 has been previously described (14). Plasmid 4878-MCP1 is constructed as follows: a 604 bp fragment containing the entire coding sequence of murine CCL2 is removed from pGEM-1 by EcoR1 restriction digestion, ends blunted using the large (Klenow) fragment of DNA polymerase I, and subcloned into a blunted KpnI site located between the Egr-1 promoter and hepatitis polyA sequences within pRB4878. All restriction and modifying enzymes are purchased from Invitrogen Life Technologies (Carlsbad, Calif.). To generate M010, the targeting plasmid 4878-CCL2 is co-transfected with HSV-1 DNA R3659 (36) using 15 μl Lipofectamine (Invitrogen). Recombinant thymidine kinase (tk) negative viruses are selected using the standard tk selection method (33), and the tk gene is repaired at its native locus, as previously described for M002 (28). Introduction of CCL2 under the control of the Egr-1 promoter into the γ₁34.5 locus, and repair of tk at its native locus is confirmed by Southern blot hybridization (data not shown).

### Example 3

### ELISA

Production of murine CCL2 by M010 is confirmed and quantified using a commercially available ELISA kit (R&D Systems, Minneapolis, Minn.), whereas IL-12 production by M002 has been demonstrated previously (28). For in vitro CCL2 production, six well plates are seeded at a confluency of 1.5×10⁵ (Vero) or 4×10⁵ (Neuro-2a) cells/well one day prior to infection with desired recombinant or control virus at a multiplicity of infection (MOI)=1 in a total volume of 0.5 ml. After 2 hr, inoculum is removed, 1 ml of growth medium is overlaid onto infected wells and plates are incubated 24 hr at 37° C. Supernates are removed, and appropriately diluted samples are analyzed by ELISA, according to the manufacturer's protocol. Experiments are performed in duplicate and the average level of cytokine production is determined, and levels detected are well within range of the kit sensitivity (between 15.6 μg/ml to 1000 μg/ml). For analysis of IL-12, IFN-γ or CCL2 production in Neuro-2a flank tumors, tumors are established as described below, until the appropriate size is achieved. Tumors are injected with 1×10⁷ pfu of either M002 or M010 in 50 μl total, or with 50 μl of serum-free DMEM. Animals are sacrificed and tumors removed at days 3, 6, 10 or 13 after treatment, weighed, and diluted 1:5 (w/v) with DMEM:F12 containing 1% FBS. Tumors are homogenized and 0.1 ml of the homogenate supernatants (i.e. 20 mg of tumor) are analyzed for IL-12, IFN-γ or CCL2 production by ELISA using commercially available kits (R&D Systems) as described above. At least 2 mice are analyzed per time point. Cytokine production is standardized to the weight of the tumor (pg/ml cytokine per 20 mg tumor).

### Example 4

### Animals

Specific pathogen-free female A/J strain mice are obtained from Charles River Laboratories and used at approximately 8 weeks of age. All animal studies are conducted in accordance with guidelines for animal use and care established by The University of Alabama at Birmingham Animal Resource Program and the Institutional Animal Care and Use Committee (IACUC protocol numbers 000203985, 000903963 and 020506278).

### Example 5

### Neuro-2a Flank Tumor Model

To establish Neuro-2a flank tumors in A/J mice, cultured Neuro-2a cells are trypsinized and seeded at 3.0×10⁶ cells, or higher, in 0.2 ml serum-free DMEM and injected subcutaneously implanted into the right flanks of 6-8 week old A/J mice. When palpable tumors are detected, typically 5-7 days after tumor cell implantation, animals are weighed and tumors are measured using vernier calipers. Due to significant heterogeneity in initial tumor volume of the animals, care is taken to evenly distribute the animals with the largest and the smallest tumors across all the treatment groups such that the average starting tumor volume for all the mice within each treatment group is approximately the same. After direct intratumoral virus injection, tumors are measured twice weekly, and animals are monitored closely for substantial weight loss (>20% initial body weight), adverse changes in grooming habits, signs of dehydration, or development of ulcerating or cannibalized tumors. Tumor growth inhibition is monitored for three weeks. If during this period any significant morbidity is observed (see criteria above), or if any given tumor measurement parameter (length, width, height) exceeded 25 mm, animals are euthanized.

### Example 6

### Statistical Analyses

Changes in tumor volume after virus (or vehicle) treatment are analyzed as follows. To adjust for heterogeneity between animals in initial tumor size, changes in tumor volume are expressed as a proportion of the volume on Day 0. Since the distribution of the outcome variable is highly skewed, a log transformation is used to both reduce the variability and make the distribution as close to a normal distribution as possible. A linear mixed model with an autoregressive covariance structure is first fit to the data from each experiment separately, and then to the combined data from both experiments. The predictors in the model are weight of the mouse, the volume, time, treatment, and the interaction between time and treatment.

### Example 7

### Histopathology

Neuro-2a flank tumors are harvested from sacrificed animals and divided into three approximately equal sections by free hand slicing. The sections are placed in a freezing cassette that is filled with Tissue-Tek OCT compound, and placed on a block of dry ice until frozen, then stored at −80° C. Frozen tumors are sectioned (8 microns thick), mounted on TEPSA-coated slides, fixed in 95% ethanol and blocked first in 5% normal rabbit serum (Vector Laboratories, Burlingame, Calif.), diluted in 1×PBS for 30 min at room temperature, followed by avidin (15 min, RT) and biotin (15 min, RT). One slide of each group is stained with hematoxylin and eosin (SurgiPath Medical Industries, Inc. Richmond, Ill.). Serial sections are reacted with rat monoclonal antibodies specific for mouse CD4 (clone GK1.5) and CD8 (clone 53-6.7) surface antigens (BD Biosciences Pharmingen, San Jose, Calif.) and the antibody binding detected using biotinylated rabbit anti-rat Ig (Vector Laboratories) followed successively with an avidin-biotin-horseradish peroxidase complex and 1% diaminobenzidine (14) (Vectastain ABC kit, Vector Laboratories). The staining is done according to the kit protocol.

### Example 8

To determine whether each of the viruses is expressing physiologically relevant levels of the respective cytokine or chemokine, culture supernates from Vero and Neuro-2a cells infected with M010 are assayed for CCL2 production by ELISA. In Vero cells, physiologically relevant levels of CCL2 are produced (37), ranging between 2-5 nM, as shown in Table 1. In Neuro-2a cells, 0.2 nM of CCL2 is produced after 48 hours infection, which is also within physiologically relevant levels (˜0.1 nM to 9 nM).

Supernates from infected cells are collected at either 24 h.p.i. (Vero) or 48 h.p.i. (Neuro-2a). n.d., not done.

TABLE 1
CCL2 production in vitro

| | R3659 | M010 (CCL2) | | M002 (IL-12) | DMEM (mock) |
|---|---|---|---|---|---|
| Vero | 0 | 2000-5000 | pM | n.d. | n.d. |
| Neuro-2a | 9.7 pM | 200 | pM | 14.2 pM | 22 pM |

### Example 9

### Combinatorial Cytokine Therapy of Neuro-2a Flank Tumors Using M002 and M010

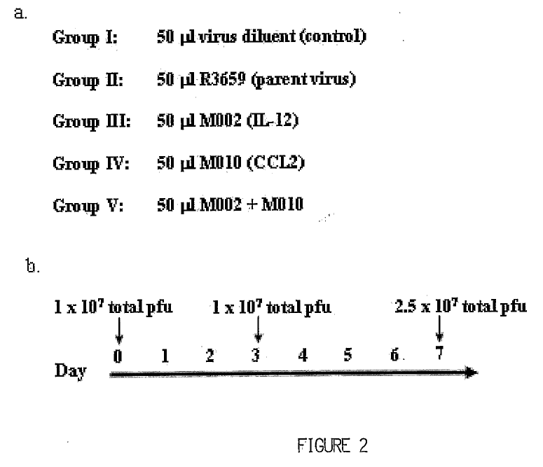

The Neuro-2a spontaneous neuroblastoma of A/J mouse origin has been previously shown to be susceptible to HSV-1 infection (38). When injected subcutaneously, Neuro-2a cells are able to establish sizable tumors on the mouse flank in a relatively short time. Neuro-2a is one of several clonal derivatives of the C-1300 spontaneous neuroblastoma of A/J mice, and has been used previously for evaluating multiple therapeutic modalities (39). The tumor exhibits low immunogenicity, including low levels of MHC I, MHC II and B7.1 expression, and is thus a stringent test for preclinical models of antitumor immunotherapy (28). The ability of M010 (HSV expressing CCL2) to inhibit growth of Neuro-2a flank tumors as compared to M002 (HSV expressing IL-12), or the parent virus R3659, is tested. In addition, Neuro-2a flank tumors are treated with both M010 and M002 to determine whether combinatorial therapy of tumors inhibited growth more effectively than therapy of tumors treated with each virus individually. To determine growth inhibition over a 21 day time period, flank tumors are measured twice weekly. The dosing scheme and treatment groups for Neuro-2a flank tumors treated with each of the viruses alone, or a combination of M002+M010, is illustrated in FIG. 2. For the combination treatment group, equivalent amounts of each virus are injected such that the total pfu administered is equal to that given in the single virus treatment groups. For example, in the combination treatment group, 5×10⁶ pfu M002+5×10⁶ pfu M010 is administered for the first two doses, which is equivalent to the first two doses of 1×10⁷ pfu each administered in the single virus groups. The flank tumor growth inhibition studies are performed in two separate experiments, and the average tumor volume for all of the mice in each treatment group (n=10) at each time point is determined. In the first experiment, groups treated with M002, M010, or a combination of the two inhibited tumor growth more effectively than groups treated with DMEM (virus diluent) only, or with the parent virus R3659 (FIG. 3). For the M002+M010 combination group, the average tumor volumes obtained in the third week of monitoring are less than the average tumor volumes obtained for either the M002 or M010 treatment groups, suggesting that combinatorial therapy is more effective than therapy with either virus alone. In the second experiment, the data varied slightly in that the average tumor volume from the M010-only treatment group did not show any growth inhibition when compared with the R3659 treatment group (data not shown). Further, growth inhibition of tumors treated with the M002+M010 combination group is more pronounced in the second experiment, with the effect appearing after the first week of the three week monitoring period (data not shown).

### Example 10

### Statistical Analysis of Inhibition of Flank Tumor Growth by Cytokine-Expressing HSV-1

To determine whether the trends observed for inhibition of tumor growth over time are of significance in the two separate experiments, the data from each experiment is submitted for statistical analysis. Since the variable of interest in each experiment is the rate of change of the mean tumor volume across time in the different treatment groups, the linear model is used to compare the slopes of the different treatment groups. This allows for the modeling of the covariance structure within the repeated measurements for each mouse. The log transformation of the mean tumor volumes for each group are then compared using Wald's test, which assumes normality.

The results show that time, weight and the interaction term are all highly significant, and suggested that the effect of treatment on the tumor volume is a function of the number of days post inoculation. This is true whether the data from the experiments are analyzed separately, or combined for the two studies. Table 2 displays the difference in log mean tumor volumes between the vehicle (DMEM) and the four other treatments at the different time points in the two experiments. In both experiments, the combination of M010 and M002 reduced tumor volume faster than treatment with either of the two viruses individually. In the first experiment, the magnitude of differences in the log tumor volumes when compared to controls is consistently higher in the combination treatment group than in the treatment groups which received only M010 or M002 (Table 2).

TABLE 2
Daily difference in log mean tumor volume produced by each
virus relative to DMEM (virus diluent negative control)

| | | | | |
|---|---|---|---|---|
| DAY 7 | 0.2212 | 0.6464 | 0.643 | 0.7098 |
| DAY 11 | 1.159 | 1.5057¹ | 1.2772¹ | 1.6661² |
| DAY 14 | 1.0302 | 1.6213² | 1.6489² | 2.167³ |
| DAY 18 | 1.5831² | 1.7301² | 1.8421² | 2.7564⁴ |
| DAY 21 | 1.5201¹ | 2.1072³ | 2.1443³ | 3.4005⁴ |
| DAY 25 | 1.2141 | 2.0352² | 2.2612³ | 3.4656⁴ |
| DAY 28 | 1.1148 | 2.2313² | 2.2448² | 3.5205⁴ |
| EXPT TWO | R3659 | M002 | M010 | M002 + M010 |
| DAY 0 | −0.0746 | 0.02662 | −0.0247 | 0.0513 |
| DAY 4 | 0.0121 | 0.266 | −0.0081 | 0.4685 |
| DAY 7 | 0.1482 | 0.3648 | 0.0833 | 0.7752¹ |
| DAY 11 | 0.2465 | 0.6438 | 0.2523 | 0.9528² |
| DAY 14 | 0.5809 | 0.9190² | 0.1828 | 0.972² |
| DAY 18 | 0.5228 | 0.9978² | 0.04016 | 0.9078² |
| DAY 21 | 0.8872¹ | 1.149 | 0.0566 | 0.8714¹ |
| DAY 25 | 1.2141² | 1.1351² | 0.1359 | 0.7147¹ |

¹0.01 < p-val < 0.05,
²0.001 < p-val < 0.01,
³0.0001 < p-val < 0.001,
⁴p-val < 0.0001

The second experiment varies from the first in that statistically significant reduction in tumor volume is not observed in the M010-only treatment group. However, as in the first experiment, the differences in tumor volumes for treatment groups which received a combination of M002 with M010 are significantly different from the control treatment group tumors. The time to achieve significant differences occurred much earlier for the combination treatment group (Day 7) versus the M002-only treatment group (Day 14).

### Example 11

### Evaluation of In Vivo Cytokine/Chemokine Expression in Neuro-2a Flank Tumors Treated with Recombinant HSV at Various Time Points Post-Tumor Therapy

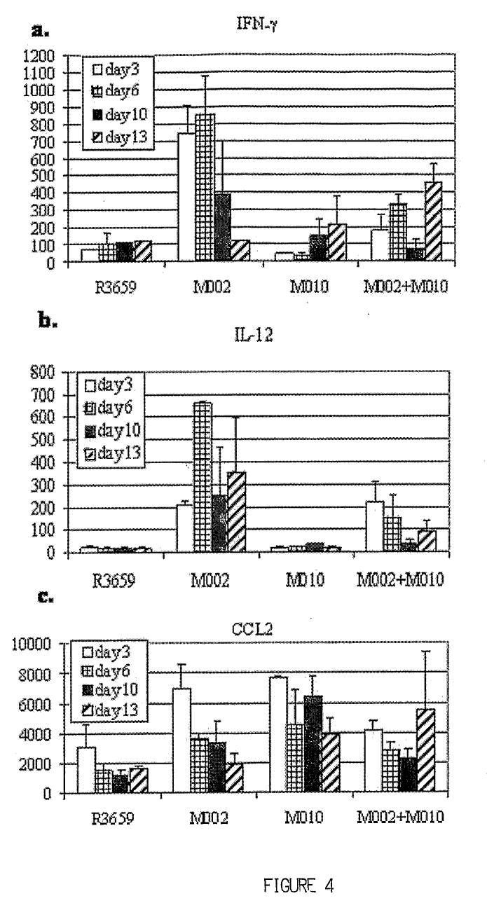

To assess in vivo the levels of IL-12 and CCL2 production over time within each treatment group, tumor-bearing mice are injected intratumorally with 1×10⁷ pfu of either R3659, M002, M010, or M002 in combination with M010. At Days 3, 6, 10 and 13 post virus-injection, two mice/group are sacrificed, tumors harvested, homogenized, and homogenates analyzed by ELISA for IL-12 or CCL2 production, as described earlier. In addition, since IL-12 enhancement of a T_{H}1-type immune response is mediated via the induction of interferon gamma (IFN-γ) secretion, IFN-γ production is also assessed by ELISA. FIG. 4 graphically illustrates the relative levels of each of these proteins over time for each treatment group. IL-12 and IFN-γ protein levels are highest in tumors treated with M002 only, with no IL-12 production in tumors treated with the parent virus R3659, or with M010 (CCL2-expressing HSV). For tumors treated with both M002 and M010, IL-12 levels are approximately the same at Day 3, but significantly more IL-12 is produced at Day 6 in the M002 only treatment group, indicating no further contribution of CCL2 to its production. For the latter time points, the error margins are significantly greater for the M002-only treatment group. Thus, it is difficult to ascertain whether the elevated IL-12 levels at these time points, as compared to the combination of M002 and M010 treatment group, are a real effect. Interestingly, the combination of M002 and M010 appears to have significantly elevated levels of IFN-γ at the last time point evaluated (Day 13) versus IFN-γ levels detected in homogenates of tumors treated with M002 only. Between 1-2 ng/mL of CCL2 is produced in Neuro-2a tumors after HSV (R3659) infection. In tumors treated with M002 or M010 only, the levels of CCL2 are similar, and remain essentially the same even after combinatorial therapy. As with the IFN-γ production, levels of CCL2 are highest in the combination group; however, the standard error is high for these samples.

### Example 12

### Evaluation of Immune Cell Infiltrates in Neuro-2a Tumors Treated with Recombinant HSV at Various Time Points Post-Tumor Therapy

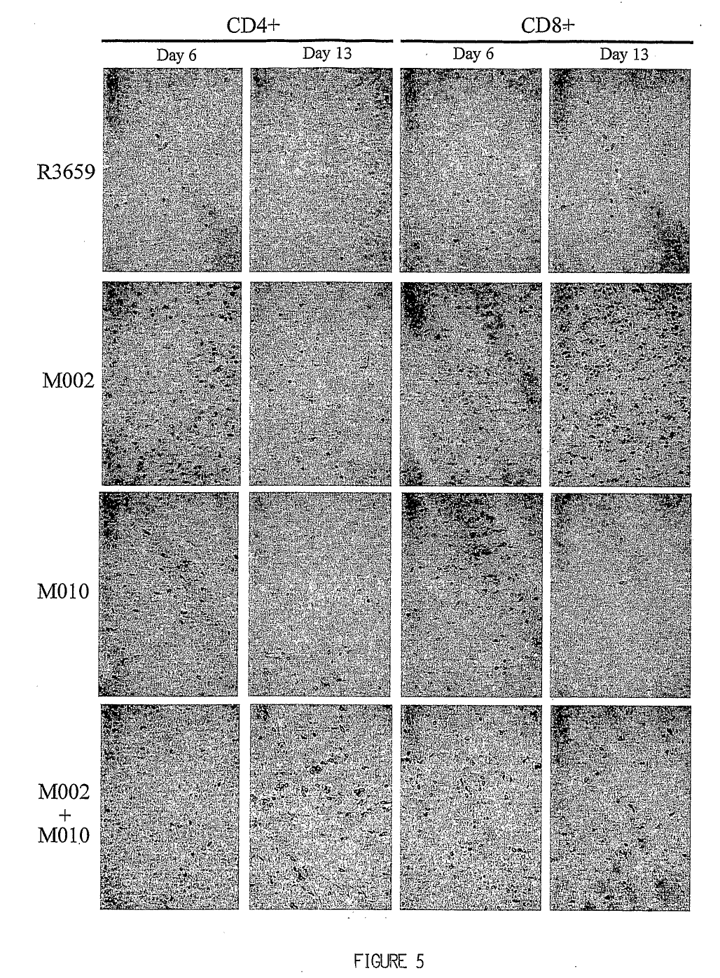

For immunohistochemical analysis of immune cell infiltrates into the tumors following HSV therapy over time, Neuro-2a flank tumors are established as before, and 1×10⁷ total pfu administered for each treatment group on Day 0. At Days 3, 6, and 13, two mice/group are euthanized, their tumors excised and frozen tissue sections are prepared and labeled with rat monoclonal antibodies specific for CD4+ and CD8+ T cells (see above). Immunohistochemical staining is performed as described in Examples herein. Representative staining patterns of tumor sections for the different treatment groups are shown in FIG. 5 for Days 6 and 13. To quantify and compare the numbers of CD4+ and CD8+ cells elicited after treatment for a given time point, representative tissue sections are photographed at 200× magnification and number of positive cells/field is determined by visual counting (Table 3).

TABLE 3
Immunohistochemical analysis of HSV-1
treated Neuro-2a flank tumors

| Treatment Group | Antibody | Day 3 | Day 6 | Day 10 | Day 13 |
|---|---|---|---|---|---|
| DMEM | CD4 | +/− | +/− | +/− | +/− |
| | CD8 | − | − | − | − |
| R3659 | CD4 | +/− | + | + | + |
| | CD8 | − | + | + | + |
| M002 | CD4 | +/− | +++ | ++ | ++ |
| | CD8 | − | ++++ | + | +++ |
| M010 | CD4 | + | ++ | ++ | ++ |
| | CD8 | + | +++ | + | +/− |
| M002 + M010 | CD4 | ++ | + | ++++ | +++ |
| | CD8 | +++ | +++ | ++ | ++ |

+/−, 0-10 positive cells/field;
+, 10-50 positive cells/field;
++, 51-100 positive cells/field
+++, 101-199 positive cells/field;
++++, >200 positive cells/field

Tumors treated with a combination of M002 and M010 elicited significant CD8+ T cell responses which are highest at Days 3 and 6 after treatment (100-200 positive cells/field), but remained significant at Days 10 and 13 post treatment (50-100 positive cells/field). Modest CD4+ T cell responses are detected at Days 3 and 6 post virus treatment (<100 positive cells/field), but are significantly enhanced by Days 10 and 13 (>100 positive cells/field), which is considerably more than any other treatment group at the latter time points. M002-only treated tumors also elicited significant CD4+ (150-160 positive cells/field) and CD8+ T cell responses (>200 positive cells/field), but they did not appear until Day 6 (<10 CD4+ or CD8+ cells/field detected at Day 3), are reduced at Day 10 (<100 positive cells/field), and then reappeared in significant levels by Day 13 (>200 CD8+ T cells/field are detected at the latest time point assessed). The M010-only treatment group elicited modest CD4+ T cell responses (<100 positive cells/field) which remained essentially unchanged for all of the time points assessed. CD8+ T cell responses elicited by treatment of tumors with M010 are initially modest (<50 positive cells/field) at Day 3, and increased significantly by Day 6 (140-150 positive cells/field). However, these responses had largely disappeared by Day 13 (<10 positive cells/field). DMEM treated tumors failed to elicit any CD8+ T cell responses and minimal CD4+ T cell responses. CD4+ and CD8+ T cell responses elicited by R3659 (parent virus) treatment also did not appear until Day 6 (<50 positive cells/field), and are essentially unchanged at the latter time points (<25 positive cells/field detected at Days 10 and 13).

### Example 13

### Construction and Evaluation of Alternate Vectors

A recombinant herpes simplex virus denoted M025 was constructed as detailed above with respect to M002 with the exception that the MIL-12 subunits within a single expression cassette are replaced with a cassette corresponding to mIL-4.

Likewise, an additional construct is formed as described previously (28) in which mCCL2 is replaced with a nucleic acid cassette coding for eotaxin and is designated M026.

The experiments of Example 9 were repeated using the Neuro-2a flanked tumor model detailed in Example 5 with the substitution of M025 for M002 and the substitution of M026 for M010. A synergistic combination treatment group of 5×10⁶ pfu M025+5×10⁶ pfu M026 administered for the first two doses yielded comparable results to that of the combination therapy of Group V depicted in FIG. 3. Administration solely of HSV expressing IL-10 or HSV expressing eotaxin while inhibiting tumor growth is not as effective as the combination therapy.

An experiment was also conducted using 5×10⁶ pfu of M002+2.5×10⁶ pfu M01+2.5×10⁶ pfu M026 in each of the first two administrations performed on Days 1 and 3 and 2½ times the doses of each delivered on Day 7. This three-component combinatorial treatment provided tumor inhibition superior to any of the combination components delivered separately. The three-component combination treatment afforded tumor volume inhibitions consistent with that seen for Group V combination treatment.

### REFERENCES

1. Shapiro W R, Shapiro J R, and Walker R W. Central nervous system. In: Abeloff, M D, Armitage J O, Lichter A S, et al., eds. Clinical Oncology, 2nd ed. New York, N.Y.: Churchill Livingstone; 2000:1103.
2. Markert J M, Gillespie G Y, Weichselbaum R R, Roizman B, and Whitley R J. Genetically engineered HSV in the treatment of glioma: a review. Rev Med Virol. 2000; 10:17-30.
3. Markert J M, Parker J N, Gillespie G Y, and Whitley R J. Genetically engineered human herpes simplex virus in the treatment of brain tumours. Herpes. 2001; 8:17-22.
4. Andreansky S S, He B, Gillespie G Y, et al. The application of genetically engineered herpes simplex viruses to the treatment of experimental brain tumors. Proc Natl Acad Sci USA. 1996; 93:11313-8.
5. Martuza R L, Malick A, Markert J M, Ruffner K L, and Coen D M. Experimental therapy of human glioma by means of a genetically engineered virus mutant. Science. 1991; 252:854-856.
6. Mineta T, Rabkin S D, Yazaki T, Hunter W D, and Martuza R L. Attenuated multi-mutated herpes simplex virus-1 for the treatment of malignant gliomas. Nat. Med. 1995; 1:938-943.
7. Kramm C M, Chase M, Herrlinger U, et al. Therapeutic efficiency and safety of a second-generation replication-conditional HSV1 vector for brain tumor gene therapy. Hum. Gene Ther. 1997; 8:2057-2068.
8. Pyles R B, Warnick R E, Chalk C L, Szanti B E, and Parysek L M. A novel multiply-mutated HSV-1 strain for the treatment of human brain tumors. Hum. Gene Ther. 1997; 8:533-544.
9. Markert J M, Malick A, Coen D M, and Martuza R L. Reduction and elimination of encephalitis in an experimental glioma therapy model with attenuated herpes simplex mutants that retain susceptibility to acyclovir. Neurosurgery. 1993; 32:597-603.
10. Chambers R, Gillespie G Y, Soroceanu L, et al. Comparison of genetically engineered herpes simplex viruses for the treatment of brain tumors in a scid mouse model of human malignant glioma. Proc. Natl. Acad. Sci. U.S.A. 1995; 92:1411-1415.
11. Chou J, Kern E R, Whitley R J, and Roizman B. Mapping of herpes simplex virus-1 neurovirulence to gamma 134.5, a gene nonessential for growth in culture. Science. 1990; 250:1262-1266.
12. Mineta T, Markert J M, Takamiya Y, et al. CNS tumor therapy by attenuated herpes simplex viruses. Gene Ther. 1994; 1 Suppl 1:S78:S78.
13. Andreansky S, Soroceanu L, Flotte E R, et al. Evaluation of genetically engineered herpes simplex viruses as oncolytic agents for human malignant brain tumors. Cancer Res. 1997; 57:1502-1509.
14. Andreansky S, He B, van Cott J, et al. Treatment of intracranial gliomas in immunocompetent mice using herpes simplex viruses that express murine interleukins. Gene Therapy. 1998; 5:121-130.
15. Kaplitt M G, Tjuvajev J G, Leib D A, et al. Mutant herpes simplex virus induced regression of tumors growing in immunocompetent rats. Journal of Neuro-Oncology. 1994; 19:137-147.
16. Yazaki T, Manz H J, Rabkin S D, and Martuza R L. Treatment of human malignant meningiomas by G207, a replication-competent multimutated herpes simplex virus 1. Cancer Res. 1995; 55:4752-4756.
17. Markert J M, Medlock M D, Rabkin S D, et al. Conditionally replicating herpes simplex virus mutant, G207 for the treatment of malignant glioma: results of a phase I trial. Gene Therapy. 2000; 7:867-874.
18. Rampling R, Cruickshank G, Papanastassiou V, et al. Toxicity evaluation of replication-competent herpes simplex virus (ICP 34.5 null mutant 1716) in patients with recurrent malignant glioma. Gene Therapy. 2000; 7:859-866.
19. Papanastassiou V, Rampling R, Fraser M, et al. The potential for efficacy of the modified (ICP 34.5(−)) herpes simplex virus HSV1716 following intratumoural injection into human malignant glioma: a proof of principle study. Gene Ther. 2002; 9:398-406.
20. Okada H, Giezeman-Smits K M, Tahara H, et al. Effective cytokine gene therapy against an intracranial glioma using a retrovirally transduced IL-4 plus HSVtk tumor vaccine. Gene Therapy. 1999; 6:219-226.
21. Wei M X, Li F, Ono Y, Gauldie J, and Chiocca E A. Effects on brain tumor cell proliferation by an adenovirus vector that bears the interleukin-4 gene. Journal of Neurovirology. 1998; 4:237-241.
22. Benedetti S, Dimeco F, Pollo B, et al. Limited efficacy of the HSV-TK/GCV system for gene therapy of malignant gliomas and perspectives for the combined transduction of the interleukin-4 gene. Human Gene Therapy. 1997; 8:1345-1353.
23. Tseng S H, Hwang L H, and Lin S M. Induction of antitumor immunity by intracerebrally implanted rat C6 glioma cells genetically engineered to secrete cytokines. Journal of Immunotherapy. 1997; 20:334-342.
24. Nishimura T, Watanabe K, Yahata T, et al. The application of IL-12 to cytokine therapy and gene therapy for tumors. Annals of the New York Academy of Sciences. 1996; 795:375-378.
25. Tahara H and Lotze M T. Antitumor effects of interleukin-12 (IL-12): applications for the immunotherapy and gene therapy of cancer. [Review] [32 refs]. Gene Therapy. 1995; 2:96-106.
26. Lamont A G and Adorini L. IL-12: a key cytokine in immune regulation. Immunol Today. 1996; 17:214-7.
27. Brunda M J, Luistro L, Rumennik L, et al. Antitumor activity of interleukin 12 in preclinical models. Cancer Chemotherapy & Pharmacology. 1996; 38:S16-21.
28. Parker J N, Gillespie G Y, Love C E, et al. Engineered herpes simplex virus expressing IL-12 in the treatment of experimental murine brain tumors. Proceedings of the National Academy of Sciences of the United States of America. 2000; 97:2208-2213.
29. Carr J A, Rogerson J, Mulqueen M J, Roberts N A, and Booth R F G. Interleukin-12 exhibits potent antiviral activity in experimental herpesvirus infections. Journal of Virology. 1997; 71:7799-7803.
30. Loetscher P, Seitz M, Clark-Lewis I, Baggiolini M, and Moser B. Monocyte chemotactic proteins MCP-1, MCP-2, and MCP-3 are major attractants for human CD4+ and CD8+ T lymphocytes. Faseb J. 1994; 8:1055-60.
31. Traynor T R, Herring A C, Dorf M E, et al. Differential roles of CC chemokine ligand 2/monocyte chemotactic protein-1 and CCR2 in the development of T1 immunity. J Immunol. 2002; 168:4659-66.
32. Luther S A and Cyster J G. Chemokines as regulators of T cell differentiation. Nat Immunol. 2001; 2:102-7.
33. Post L E and Roizman B. A generalized technique for deletion of specific genes in large genomes: alpha gene 22 of herpes simplex virus 1 is not essential for growth. Cell. 1981; 25:227-232.
34. Jenkins F and Roizman B. Herpes simplex virus 1 recombinants with noninverting genomes frozen in different isomeric arrangements are capable of independent replication. Journal of Virology. 1986; 59:494-9.
35. Lagunoff M and Roizman B. The regulation of synthesis and properties of the protein product of open reading frame P of the herpes simplex virus 1 genome. J. Virol. 1995; 69:3615-3623.
36. Lagunoff M and Roizman B. Expression of a herpes simplex virus 1 open reading frame antisense to the gamma 134.5 gene and transcribed by an RNA 3′ coterminal with the unspliced latency-associated transcript. J. Virol. 1994; 68:6021-6028.
37. Yoshimura T, Robinson E A, Tanaka S, Appella E, and Leonard E J. Purification and amino acid analysis of two human monocyte chemoattractants produced by phytohemagglutinin-stimulated human blood mononuclear leukocytes. J Immunol. 1989; 142:1956-62.
38. Lopez C. Genetics of natural resistance to herpesvirus infections in mice. Nature. 1975; 258:152-153.
39. Macklis J D and Madison R D. Neuroblastoma grafts are noninvasively removed within mouse neocortex by selective laser activation of intracellular photolytic chromophore. Journal of Neuroscience. 1991; 11:2055-2062.
40. Kikuchi T, Joki T, Saitoh S, et al. Anti-tumor activity of interleukin-2-producing tumor cells and recombinant interleukin 12 against mouse glioma cells located in the central nervous system. Int J. Cancer. 1999; 80:425-30.
41. Parney I F, Farr-Jones M A, Chang L J, and Petruk K C. Human glioma immunobiology in vitro: implications for immunogene therapy. Neurosurgery. 2000; 46:1169-77; discussion 1177-8.
42. Dzenko K A, Andjelkovic A V, Kuziel W A, and Pachter J S. The chemokine receptor CCR2 mediates the binding and internalization of monocyte chemoattractant protein-1 along brain microvessels. J. Neurosci. 2001; 21:9214-23.
43. Sakai Y, Kaneko S, Nakamoto Y, et al. Enhanced anti-tumor effects of herpes simplex virus thymidine kinase/ganciclovir system by codelivering monocyte chemoattractant protein-1 in hepatocellular carcinoma. Cancer Gene Ther. 2001; 8:695-704.
44. Tsuchiyama T, Kaneko S, Nakamoto Y, et al. Enhanced antitumor effects of a bicistronic adenovirus vector expressing both herpes simplex virus thymidine kinase and monocyte chemoattractant protein-1 against hepatocellular carcinoma. Cancer Gene Ther. 2003; 10:260-9.
45. Russell S W and Gillespie G Y. Nature, function and distribution of inflammatory cells in regressing and progressing Moloney sarcomas. J Reticuloendothel Soc. 1977; 22:159-68.
46. Gillespie G Y, Hansen C B, Hoskins R G, and Russell S W. Inflammatory cells in solid murine neoplasms. IV. Cytolytic T lymphocytes isolated from regressing or progressing Moloney sarcomas. J Immunol. 1977; 119:564-70.
47. Gillespie G Y and Russell S W. Level of activation determines whether inflammatory peritoneal and intratumoral macrophages will promote or suppress in vitro development of cytolytic T lymphocyte activity. J Reticuloendothel Soc. 1980; 27:535-45.
48. Gillespie G Y and Barth R F. Cyclic variations in cell-mediated immunity to skin allografts detected by the technetium-99m microcytotoxicity assay. Cell Immunol. 1974; 13:472-83.

Any patents or publications mentioned in this specification are herein incorporated by reference to the same extent as if each individual publication is specifically and individually indicated to be incorporated by reference. In particular, U.S. Pat. No. 6,764,675 is incorporated by reference herein.

One skilled in the art will readily appreciate that the present invention is well adapted to carry out the objects and obtain the ends and advantages mentioned, as well as those inherent therein. The apparatus and methods described herein are presently representative of preferred embodiments, exemplary, and not intended as limitations on the scope of the invention. Changes therein and other uses will occur to those skilled in the art. Such changes and other uses are encompassed within the spirit of the invention as defined by the scope of the claims.

1. A conditionally replicating, aneurovirulent recombinant herpes simplex virus, comprising:
a nucleic acid encoding an expressible chemokine; and
an expression control element operably linked to the nucleic acid.

2. The virus of claim 1 wherein the nucleic acid encoding a chemokine is inserted in a γ₁34.5 locus of a herpes simplex virus genome.

3. The virus of claim 1 wherein the chemokine is a C-C chemokine.

4. The virus of claim 1 wherein the chemokine is selected from the group consisting of: RANTES, MIP-10, and MIP-10, MCP-2, MCP-3, MCP-4, eotaxin, 1-309, HCC-1, HCC-2, and HCC-4.

5. The virus of claim 1 wherein the chemokine is CCL2.

6. A therapeutic composition comprising:
a first conditionally replicating, aneurovirulent recombinant herpes simplex virus, the first virus comprising a first nucleic acid encoding a first expressible cytokine and a first expression control element operably linked to the first nucleic acid;
a second conditionally replicating, aneurovirulent recombinant herpes simplex virus, the second virus comprising a second nucleic acid encoding a second expressible cytokine and a second expression control element operably linked to the second nucleic acid; and
a pharmaceutically acceptable carrier.

7. The composition of claim 6 wherein the first cytokine is selected from the group consisting of: GM-CSF, IL-1α, IL-1β IL-2, IL-3, IL-4, IL-5, IL-6, IL-7, IL-10, IL-15, IL-23, IL-24, IFN-α, IFN-β, IFN-γ, TNFα, and TNFβ.

8. The composition of claim 6 wherein the first cytokine is IL-12.

9. The composition of claim 6 wherein the second cytokine is a chemokine.

10. The composition of claim 9 wherein the chemokine is selected from the group consisting of: a C-X-C chemokine lacking an ELR motif a CC chemokine, and a C chemokine.

11. The composition of claim 9 wherein the chemokine is selected from the group consisting of: IP-10, Mig, I-TAC, SDF-1, BCA-1, RANTES, MIP-1α, MIP-1β, MCP-2, MCP-3, MCP-4, eotaxin, I-309, HCC-1, HCC-2, HCC-4 and lymphotactin.

12. The composition of claim 9 wherein the chemokine is CCL2.

13. A therapeutic composition comprising:
a first conditionally replicating, aneurovirulent recombinant herpes simplex virus comprising a first nucleic acid which upon expression within a host cell increases availability of an immunoresponsive cell for activation, the cell selected from the group consisting of: CD4+, CD8+, NK, dendritic cell, and a combination thereof;
a second recombinant herpes simplex virus comprising a second nucleic acid which upon expression in the host cell or a second host cell activates the immunoresponsive cell selected from the group consisting of: CD4+, CD8+, NK, dendritic cell and combinations thereof; and
a pharmaceutically acceptable carrier.

14. A method of inhibiting tumor cell growth comprising:
introducing a therapeutically effective amount of a composition according to claim 6 into a tumor of an individual, such that the first virus infects a first cell and the second virus infects the first cell or a second cell, such that a first virally encoded cytokine is produced in the first cell and a second virally encoded cytokine is produced in the first cell or the second cell, the first and second cytokines effective to enhance an immune response in the individual that inhibits tumor cell growth.

15. A method inhibiting tumor cell growth comprising:
administering a first dose of said first conditionally replicating, aneurovirulent recombinant herpes simplex virus of claim 13, said first recombinant herpes simplex virus comprising a first nucleic acid that upon expression increases availability of a cell for activation, the cell selected from the group consisting of: CD4+, CD8+, NK, dendritic cell, and a combination thereof; and
administering a second dose of said second conditionally replicating, aneurovirulent recombinant herpes simplex virus of claim 13, said second recombinant herpes simplex virus comprising a second nucleic acid that upon expression activates a cell selected from the group consisting of: CD4+, CD8+, NK, dendritic cell, wherein said first nucleic acid and said second nucleic acid are operably linked to expression control elements.

16. The method of claim 15 wherein the first and second doses are administered simultaneously as a single composition.

17. The method of claim 15 wherein the first and second doses are administered sequentially.

18. The method of claim 15 wherein the first and second herpes simplex virus are administered in a ratio ranging from 1000:1-1:1000, inclusive.

19. The method of claim 15 wherein the total number of plaque forming units in the first and second doses combined ranges between 1×10²-1×10⁵⁰ plaque forming units.

20. A method of inhibiting tumor cell growth which comprises administering to a tumor within an individual an effective amount of a virus as claimed in claim 1.

21-22. (canceled)
